(19) United States
(12) Patent Application Publication
(10) Pub. No.: US 2002/0088062 A1
(43) Pub. Date: Jul. 11, 2002

(54) HAIR COLOURING COMPOSITIONS AND THEIR USE

(76) Inventor: DOMINIC PRATT, BERKSHIRE (GB)

Correspondence Address: S T MURPHY, THE PROCTER & GAMBLE COMPANY, 11510 REED HARTMAN HIGHWAY, SHARON WOODS TECHNICAL CENTER PATENT DIVISION, CINCINNATI, OH, 45241, US

(21) Appl. No.: 09/424,474

(22) PCT Filed: May 22, 1998
(86) PCT No.: PCT/US98/10490

(30) Foreign Application Priority Data
May 23, 1997 (GB) 9710759.3
May 23, 1997 (GB) 9710754.4
May 23, 1997 (GB) 9710756.9
May 23, 1997 (GB) 9710757.7
May 23, 1997 (GB) 9710761.9

(51) Int. Cl. A61K 7/13

(52) U.S. Cl. 8/405; 8/406; 8/421

(57) ABSTRACT

Hair coloring compositions comprising: (i) one or more developers selected from amino aromatic systems capable of being oxidized and thereafter undergoing a single nucleophilic attack, and (ii) one or more couplers selected from (A) (1) naphthols having an active leaving group in the para-position relative to the —OH group, or (2) phenols with an active leaving group which is hydrogen which is para to the —OH group, (B) 1,3-diketones, or (C) pyrazolones, such that in the presence of an oxidizing agent the or each developer reacts with the or each coupler substantially only at the position having the active leaving group.

This invention relates to new compositions for colouring hair and to methods of using these compositions in hair colouring processes.

Compositions are well known for providing various colours to hair by dyeing, either for changing a natural hair colour or for covering grey hair. Such compositions comprise various aromatic compounds, commonly known as developers (also known as precursors or primary intermediates), together with various other aromatic compounds, commonly known as couplers. These are referred to as oxidative hair colouring agents because they require an oxidising agent for formation of colour.

The developers are generally 1,4-disubstituted compounds, most commonly 1,4-diaminobenzene compounds and the couplers can also be disubstituted benzene compounds, such as 1,3-disubstituted benzene compounds. The range of structures of couplers is much more varied than that of developers.

In use, the compounds are subjected to oxidising conditions under which the developers and couplers react to form colour. It is generally believed that this occurs by means of a stepwise sequence in which developer molecules are activated by oxidation and react with couplers to form reactive dimers. These then continue reacting to form coloured trimers, which do not react further. It is believed that the monomeric developers and couplers, and to a lesser extent the dimers, diffuse into the hair shaft during the course of the reaction, which is fairly slow. When inside the hair shaft, the dimers react further to form trimers which are too large to diffuse out easily and are thus trapped, colouring the hair.

Standard products normally include several different developers and several different couplers, for instance up to 5 developers and 5 or more couplers. It is generally accepted that 10 to 12 different compounds are required to achieve the full range of colours.

Unfortunately, this system, although effective and commercially successful, has various drawbacks.

Firstly, developers can react with each other as well as with couplers and the reactive dimers can react with developers and couplers. Thus the reaction chemistry is undefined and it is not possible to predict with any precision the compounds which will be present in the hair at the end of the colour-forming reaction. The exact composition of the coloured molecules formed in the hair can vary from process to process according to the prevailing conditions. Therefore the colours eventually obtained can vary between applications.

A further drawback is that of fading of colour over time. A contributory factor in fading is lack of wash-fastness. The trimeric coloured molecules produced tend to be soluble in water and in other solvents. Consequently they tend to leach out of the hair after repeated washing and applications of, for instance, hairspray and other hair care products. This leads to gradual fading or changing of the applied colour. The action of other factors such as ultraviolet light, combing and perspiration also affects the colour.

This is a problem which has existed with commercial products for several years and which has not yet been solved.

A further problem arises from the fact that the oxidation reaction involves two steps. Thus sufficient oxidising agent must be present in the hair dye composition to induce the two oxidation steps. The presence of large amounts of oxidising agent can have undesirable effects on skin and hair.

GB 1,025,916 discloses certain developers and couplers of different types. It describes developers which are N,N-disubstituted phenylene diamine derivatives. Three classes of coupler are described. A blue colour is said to arise from phenol derivatives. A yellow colour is said to arise from certain defined R—CO—CH₂—COR derivatives and a red colour is said to arise from certain pyrazolone derivatives.

These combinations are advantageous in that the developers do not react with themselves and can react with each coupler in only one way, so that the final chemistry of the dye obtained is closely defined and highly predictable.

GB 1,025,916 describes mixing pairs of couplers, for instance red with blue, blue with yellow, etc so as to obtain shades between the colours which would be obtained with either of the couplers used individually. We have found that the dye combinations described in GB 1,025,916 give inferior fade resistance and wash fastness.

We are not aware that a system of the type described in GB 1,025,916 has ever been commercialised.

It would be desirable to be able to provide a hair colouring system in which the final colour produced is predictable and controllable but which also gives rise to long term wash fastness and fade resistance.

According to a first aspect of the present invention we provide a hair colouring composition comprising

(i) one or more developers selected from amino aromatic systems capable of being oxidised and thereafter undergoing a single electrophilic attack, and

(ii) one or more couplers selected from

(A) (1) naphthols having an active leaving group in the para-position relative to the OH group,

or (2) phenols of the formula

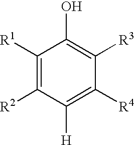

in which the H para to the OH group is an active leaving group and R¹, R², R³ and R⁴ are each independently selected from the group consisting of H, OH, methyl, ethyl, n-propyl, i-propyl, t-butyl, NH₂, CO₂H, CO₂R and COR, in which R is substituted or unsubstituted alkyl or alkenyl,

(B) 1,3-diketones (1) containing the group

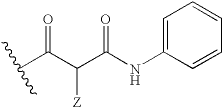

in which the N-phenyl group has no carboxy substituents,

or (2) containing the group

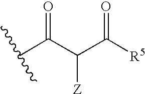

in which R⁵ contains an aryl group, and

or (3) of the formula

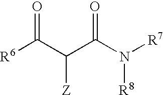

in which R⁶ is selected from the group consisting of methyl, ethyl, n-propyl, i-propyl, t-butyl and phenyl and R⁷ and R⁸ are each independently selected from the group consisting of methyl, ethyl, n-propyl, hydroxymethyl and hydroxypropyl,

or (4) of the formula

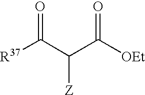

in which R³⁷ is C₄ alkyl,

in all of which Z is an active leaving group, and

(C) compounds of the formula

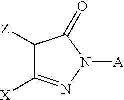

in which X is a non-leaving substituent and in which Z is an active leaving group and in which A is H or methyl,

and such that in the presence of an oxidising agent the or each developer reacts with the or each coupler substantially only at the position having the active leaving group

and provided that the composition includes at least one coupler (B) and/or at least one coupler or (C).

Thus the compositions of the invention include at least one developer (i) and at least one coupler (ii). The coupler (ii) includes at least one coupler (B) and/or at least one coupler (C), i.e. one or more couplers (B) or one or more couplers (C) or at least one of each. Coupler (B) can be selected from couplers (B)(1), (B)(2) and (B)(3). The composition may also contain at least one coupler (A), which can be selected from couplers (A)(1) and (A)(2).

In the invention the developer is an amino aromatic compound which has a structure such that it is capable of being oxidised by an oxidising agent. The structure is also such that the oxidised developer is capable of undergoing electrophilic attack by one other molecule. In other words, the structure of the developer is such that it reacts substantially only at one position, which is normally an amine. Suitable developers of this type include amino aromatic systems in which there is only one primary amine group, at which reaction occurs, other amine and other reactive groups being protected by blocking substituents.

The defined types of coupler are such that in the presence of an oxidising agent they each couple with the developer at only one position so as to produce only one resulting coloured dimer. The defined developer also reacts only at one position.

Formation of colour is, we believe, by reaction of one developer molecule with one coupler molecule to form a coloured dimer. The dimers are not reactive and no further reaction takes place. Consequently the formation of colour is extremely efficient. Further, the coloured molecules formed are very pure. With knowledge of the developer and coupler molecules present in the reaction system it is possible to predict closely and accurately the final combination of coloured molecules, and hence the final overall colour, which will be produced. These are significant advantages in comparison with standard oxidative colouring systems.

Further, certain of the coloured dimers formed have significantly reduced water-solubility in comparison with the trimers formed in standard oxidative colouring systems, which assists in achieving increased wash fastness in comparison with standard oxidative colouring systems. In some cases the structure of the developers and couplers is such that they show a greater degree of diffusion into the hair shaft than the colouring agents in known systems, and consequently lead to improved fade resistance.

We have also found that the reaction between the defined developer and the defined couplers is potentially very fast and efficient. This gives potential for hair colouring systems which do not require the long exposure times which have up to now been standard in order to produce a deep and lasting coloration.

Additionally, we believe that the wash fastness of dyes containing the defined couplers in combination with the defined developer is improved in comparison with those of GB 1,025,916.

Some suitable coupler molecules (A), (B) and (C) are known from the field of photography, as are some developers. When they have reacted with a developer molecule the couplers (A) give a cyan colour, the couplers (B) give a yellow colour and the couplers (C) give a magenta colour.

Each coupler contains a moiety of a specific formula which is such that it has an active leaving group (represented as Z in formulae in which it is not always H) at a defined site. By an “active leaving group” we mean any group which can be removed (under the conditions prevailing during the hair-colouring process) so that the developer reacts at that position in the coupler molecule. The bond formed between the coupler and developer molecule is thus formed at the site of the active leaving group. Examples of active leaving groups are H, PhO, Cl, Br, alkoxy (RO) such as phenoxy PhO, and RS— in which R is alkyl or aryl, but any leaving group which leaves during the reaction so as to allow coupling between developer and coupler is suitable.

Couplers (A) give a cyan colour. The particular shade or intensity of colour can be varied by varying the substituents of the phenol molecule (in cases (A)(2)) or naphthol molecule (in cases (A)(1)). It has an active leaving group in the para-position relative to the OH group. In case (2) this is an active proton, ie the aromatic ring is unsubstituted in the para-position and other substituents on the ring are not such as to reduce the reactivity at this position. In case (1) it may also be an active proton, or another active leaving group.

Couplers (A) may be (1) naphthols having an active leaving group in the Para-position relative to the OH group. In this case they generally have the formula I, as follows:

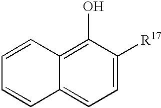

in which R¹⁷ is H, OH, —CO₂H, —CO₂R, F, Cl, Br, —CN, —NO₂, —CF₃, cycloalkyl, alkenyl, cycloalkenyl, aryl, alkaryl, aralkyl, —NH₂, —NHR, —NHCOR, —NR₂, —NHCOR, —R′NHCOR, —CONHR, R′CONHR, —R′OH, —SO₂R, SO₂NHR, —R′SO₂R, —R′SO₂NHR, —SO₃H, —OR, —R′OR or —COR, in any of which R is H, alkyl, cycloalkyl, alkenyl, cycloalkenyl, aryl, alkaryl or aralkyl, and R′ is alkylene, alkenylene, cycloalkylene, cycloalkenylene, arylene, alkarylene or aralkylene, or substituted versions of any of these. R¹⁷ may for instance be alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR. R¹⁷ may include an aryl group. It is preferred that R¹⁷ is H.

In this specification, unless otherwise stated alkyl and alkenyl are usually C₁₋₈, often C₁₋₄, cycloalkyl and cycloalkenyl are usually C₅₋₈, often C₆, aryl or ar- is usually phenyl or naphthyl and the alk-moiety in alkaryl is usually C₁₋₆, often C₁₋₃.

Preferably R¹ is H. In other suitable couplers R¹⁷ is

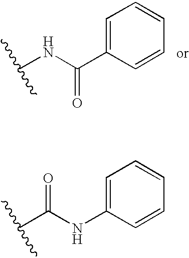

Suitable couplers (A)(1) thus have the formula II or III, as follows:

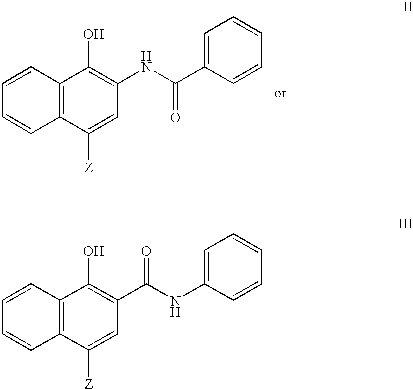

When coupler (A) is a naphthol (1) it is preferred that it has no solubilising substituents (other than Z), in particular no —COOH or —OH substituents. Coupler (A) (1) is preferably an unsubstituted naphthol.

We find that couplers of the formula II, in particular when R¹7 is H, and especially when Z is H, have an advantageous combination of properties for improving wash fastness whilst allowing rapid colouring. We believe this is because their molecules have a structure such that as monomers they are small enough to diffuse into the hair shaft but as dimers they are trapped within the hair shaft. Further, the solubility of the dimers is such that they are not easily washed out during subsequent hair treatment processes. They are particularly advantageous for colouring damaged hair (eg. hair which has been previously coloured, permed and/or bleached).

In particular these cyan couplers (A) (1) of the invention show improved fade resistance properties, especially on damaged hair, in comparison with those of GB 1,025,916.

In the above formulae Z is any active leaving group. Suitable examples are H, PhO, Cl and Br but any other groups which react similarly (under the conditions of the hair-colouring reaction) may be used. If Z is PhO, Cl or Br the reactivity of the coupler can tend to be increased in comparison with couplers in which Z is H.

In any of the above formulae I to III, the defined groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different.

If phenyl groups are substituted, mono substitution is preferred. Preferably the groups are unsubstituted unless otherwise stated.

Alternatively, the couplers (A) can be selected from a specific defined group of phenols (2) having the formula IV

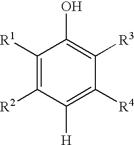

in which the H para to the OH group is an active leaving group and R¹, R², R³ and R⁴ are each independently selected from the group consisting of H, OH, methyl, ethyl, n-propyl, i-propyl, t-butyl, NH₂, CO₂H, CO₂R and COR, in which R is substituted or unsubstituted alkyl or alkenyl.

In this case the active leaving group is always H. Preferred couplers of this type include 3-aminophenol.

These preferred couplers (A) (2) also show particularly good performance on damaged hair. They demonstrate good colour uptake and good wash fastness. They also show good colour uptake and wash fastness on undamaged hair.

Yellow couplers (B) are 1,3-diketones. They may have one of two formulae. A first set (1) contain the group

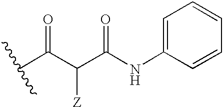

and generally have the formula V:

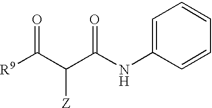

In these couplers the N-phenyl group may contain any non-interfering substituent, that is any group which does not hinder the colouring reaction between developer and coupler, with the exception that it may not contain any carboxy substituents. We find that molecules of this general type but having carboxy substituents, for instance those described in GB 1,025,916, exhibit reduced wash fastness in comparison with the couplers of the above structure.

Preferably also the N-phenyl contains no hydroxy substituents, and particularly preferably it contains no solubilising substituents. Most preferably it is unsubstituted.

The group R⁹ may be H, alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR or —OR. In these groups R can be H, alkyl, cycloalkyl, alkenyl, cycloalkenyl, aryl, alkaryl or aralkyl and R′ is alkylene, cycloalkylene, alkenylene, cycloalkenylene, arylene, alkarylene or aralkylene. Substituted versions of any of these can be used. Suitable substituents include OH, —OR, Cl, Br, F, —co₂H, —co₂R, —NH₂ and —COR. R⁹ can for instance be alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR. Preferably R⁹ is alkyl, alkenyl, alkaryl, alkenaryl, aralkyl or aralkenyl. More preferably R⁹ is alkyl, aralkyl or alkaryl. Particularly preferred R⁹ groups are phenyl and C₁₋₃ alkyl, in particular ethyl and, especially, methyl.

Yellow couplers (B)(2) contain the group

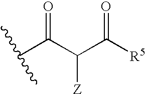

and in particular may have the following formula VI

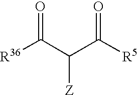

in which R⁵ contains an aryl group. Preferably R⁵ contains a phenyl group. It is preferred that the aryl, preferably phenyl, group does not contain any carboxy substituents. More preferably it does not contain any hydroxy substituents and particularly preferably it contains no solubilising substituents. In particular, R⁵ contains an unsubstituted phenyl group.

R³⁶ may for instance be alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR or —OR. It is preferably methyl.

A preferred group R⁵ is

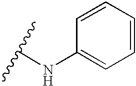

We find that couplers of this formula also have a particularly advantageous combination of properties for improving wash fastness whilst allowing rapid colouring, especially of damaged hair. In particular, they show improved wash fastness over the couplers of GB 1,025,916. We believe this is in part because of the presence of the aryl group in group R⁵, which increases the size of the final dimer trapped within the hair shaft.

In the above formulae V and VI, Z may be any of the leaving groups indicated for Z in coupler (A) above.

Further preferred yellow couplers (B) (3) have the formula VII

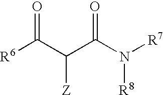

in which R⁶ is selected from the group consisting of methyl, ethyl, n-propyl, i-propyl, t-butyl and phenyl and R⁷ and R⁸ are independently selected from the group consisting of methyl, ethyl, n-propyl, hydroxymethyl and hydroxypropyl. Couplers (B)(3) are particularly advantageous for the coloration of undamaged as well as damaged hair. On undamaged hair they show fast colour uptake without loss of wash fastness. They also show good wash fastness and fade resistance on damaged hair.

In formula VII, Z may be any of the leaving groups indicated for Z in coupler (A) above. Preferably Z is H.

A further coupler (B)(4) is of the formula XXX

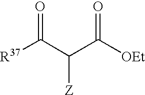

in which R³⁷ is C₄ alkyl, preferably t-butyl.

In the above formulae V, VI, VII and XXX, the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂, NHCH₃, SO₃H, C₁₋₃ alkyl such as ethyl or propyl and CONHR in which R is preferably C₁₋₃ alkyl. Phenyl groups in particular may contain one or more substituents which are the same or different. If phenyl groups are substituted, mono substitution is preferred. Preferably groups are unsubstituted unless otherwise stated.

Magenta couplers (C) have the formula VIII

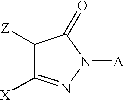

in which X is a non-leaving substituent and in which Z is an active leaving group and in which A is H or methyl.

Preferred couplers (C) have the formula IX

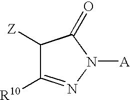

in which R¹⁰ is selected from the group consisting of H, methyl, ethyl, n-propyl, i-propyl, t-butyl and phenyl.

In the above formulae VIII and IX, Z may be any of the leaving groups indicated for Z in coupler (A) above.

In the above formulae VIII and IX, the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂, NHCH₃, SO₃H, C₁₋₃ alkyl such as ethyl or propyl and CONHR in which R is preferably C₁₋₃ alkyl. Phenyl groups in particular may contain one or more substituents which are the same or different. If phenyl groups are substituted, mono substitution is preferred. Preferably groups are unsubstituted unless otherwise stated.

Couplers of the formulae VIII and IX are particularly suitable for colouring undamaged hair and give good fade resistance and colour uptake on undamaged as well as damaged hair.

Specific examples of couplers (A) include α-naphthol, 3-aminophenol and the compounds having the following structural formulae:

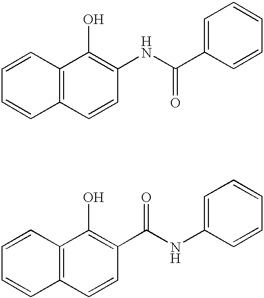

Specific examples of couplers (B) include benzoylacetanilide, acetoacetanilide, N,N-diethyl and N,N-dimethyl acetoacetamide and the compounds of the formulae

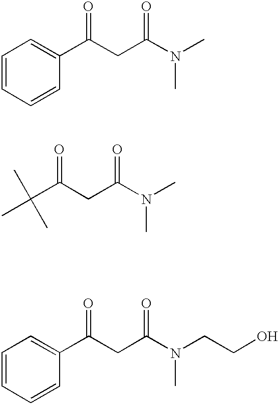

Specific examples of couplers (C) include the pyrazolone of structural formula:

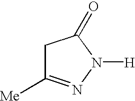

and compounds having the same formula except that Me is replaced by H or phenyl and/or H is replaced by methyl.

The composition of the invention may optionally contain additional couplers.

For instance it may contain additional cyan couplers of the general formula X

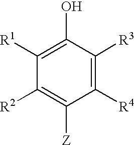

in which R¹ to R⁴ may be independently selected from H, OH, —CO₂H, —CO₂R, F, Cl, Br, —CN, —NO₂—, CF₃, cycloalkyl, alkenyl, cycloalkenyl, aryl, alkaryl, aralkyl, —NH₂, —NHR, —NHCOR, —NR₂, —NHCOR, —R′NHCOR, —CONHR, R′CONHR, —R′OH, —SO₂R, SO₂NHR, —R′SO₂R, —R′SO₂NHR, —SO₃H, —OR, —R′OR or —COR, in any of which R is H, alkyl, cycloalkyl, alkenyl, cycloalkenyl, aryl, alkaryl or aralkyl, and R′ is alkylene, alkenylene, cycloalkylene, cycloalkenylene, arylene, alkarylene or aralkylene, or substituted versions of any of these. This formula encompasses couplers (A) discussed above. Thus in addition to those specific couplers (A) the composition may contain further cyan couplers provided they have the formula X as defined.

In preferred compositions of the invention additional yellow couplers are included. These are compounds of the formula XI as follows:

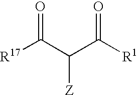

in which R¹⁷ and R¹⁸ are each, independently, selected from H, alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR and —OR. In these groups R can be H, alkyl, cycloalkyl, alkenyl, cycloalkenyl, aryl, alkaryl or aralkyl and R′ is alkylene, cycloalkylene, alkenylene, cycloalkenylene, arylene, alkarylene or aralkylene. Substituted versions of any of these can be used. Suitable substituents include OH, —OR, Cl, Br, F, —CO₂H, —CO₂R, —NH₂ and —COR.

Thus this formula broadly encompasses some of the couplers from which the essential couplers of the invention can be chosen but additionally includes further couplers.

Preferably at least one of R¹⁷ and R¹⁸ contains an aryl group.

Some preferred additional yellow couplers are of the formula XII as follows:

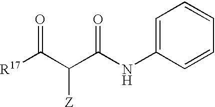

In this formula it can be preferred that R¹⁷ is phenyl.

Preferred compositions of the invention may additionally include further magenta couplers. These are pyrazolone derivatives. In particular they contain the group

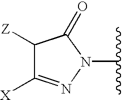

in which Z is an active leaving group and X is an active leaving group or a non-leaving substituent.

Normally X is a non-leaving substituent and they are of the formula XIII, as follows:

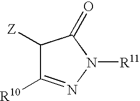

in which R¹⁰ may be any of the groups as listed for R¹⁰ above. When R¹¹ is H or Me then the couplers fall within the scope of the formula discussed above in the context of the essential components of the invention. However R¹⁰ or R¹¹ may also be selected from alkyl, alkenyl, cycloalkyl, cycloalkenyl, aryl, alkaryl, aralkyl, —R′NHCOR, —R′CONHR, —R′OH, —R′SO₂R, —R′SO₂NHR or —R′OR in which R is H, alkyl, cycloalkyl, alkenyl, cycloalkenyl, aryl, alkaryl or aralkyl, and R′ is alkylene, cycloalkylene, alkenylene, cycloalkenylene, arylene, alkarylene or aralkylene (or substituted versions of any of these). Suitable substituting groups include OH, —OR, Cl, Br, F, —CO₂H, —CO₂R, —NH₂, and —COR. They may be selected from for instance alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR.

R¹¹ is advantageously lower (C₁₋₄) alkyl such as methyl, ethyl, n-propyl, i-propyl or t-butyl, or substituted or unsubstituted phenyl. When it is phenyl, it can be preferred that it is a phenyl free of solubilising substituents, in particular —COOH and —OH substituents. m-SO₃H and p-SO₃H substituents can be used. When R¹¹ is phenyl it can preferably be unsubstituted. R¹¹ can also preferably be methyl.

Preferably R¹⁰ is methyl. It may alternatively be —NHR or —NHCOR in which R is any of the groups listed for R in coupler (A) above. R¹¹ is preferably phenyl. It is preferred that it is a phenyl free of solubilising substituents, in particular —COOH and —OH substituents.

R¹⁰ can be in particular

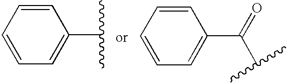

Magenta couplers in which R¹⁰ is H, lower (C₁₋₄) alkyl such as methyl, ethyl, n-propyl, i-propyl or t-butyl, or substituted or unsubstituted phenyl, in particular H or methyl, are preferred. Magenta couplers in which R¹¹ is methyl are also preferred.

Preferably any couplers in the composition are only of the defined types (A), (B) and (C). However, some compositions of the invention do additionally contain cyan and/or yellow and/or magenta couplers not of these defined types but according to the definitions of additional couplers above (formulae X to XIII) in combination with one or more couplers of types (A), (B) and (C). Preferred systems include at least one of each of the couplers (A), (B) and (C).

In preferred compositions coupler (A) is included in combination with at least one coupler of type (B) and/or at least one coupler of type (C). Alternative preferred compositions contain at least one coupler of type (B) in combination with couplers of type (A) and/or couplers of type (C).

Particularly preferred compositions contain at least one coupler of type (A), at least one coupler of type (B) and at least one coupler of type (C). In some preferred compositions not more than two or even only one compound of any or all of the types (A), (B) and (C) is included. If additional couplers of the formulae X to XIII are included, then preferably not more than three, in particular only one or two, of each of the additional cyan, yellow and magenta couplers is included. In particular not more than three or even only one or two couplers of each colour type (cyan, yellow or magenta) are included.

A particular advantage of the use of the couplers of the invention is that it is possible to obtain the full range of colours using just three specific types of coupler and one type of developer.

This allows the couplers to be supplied in the form of a mixture having the required amounts of each type of coupler to develop whichever colour is desired. Alternatively, it also allows supply of the coupler materials separately packaged so that the consumer can control the eventual colour which is obtained by mixing the correct amounts of each coupler to form the composition.

Usually coupler is present in the composition in a total amount of from 0.01% to 5 or 10% by weight based on total weight of composition applied to the hair.

Preferably total amounts of coupler are at least 0.01 wt %, often at least 0.1 or 1%. Preferably they are not more than 6%, and can in some preferred compositions be present in amounts of up to 3%, for instance not more than 2.5%. Couplers of types (A) and (C) (and other cyan and magenta couplers as discussed above) can be used in particularly low amounts. For instance couplers of type (A) (and other cyan couplers discussed above) may be used in amounts, by weight based on total weight of composition applied to the hair, of from 0.001 to 1%, preferably 0.04 or 0.005 to 0.5%, for instance not more than 0.005 wt %.

Couplers of type (C) (and other magenta couplers discussed above) can be used in amounts of from for instance 0.01 to 2 or 4%, preferably 0.03 to 3 or 2%, and in some compositions not more than 1 or 0.5%. Couplers of type (B) (and other yellow couplers discussed above) are often used in larger amounts, for instance from 0.05 to 3 or 4 wt % (but in some cases up to 5 or 6%), for instance 0.1 to 2 or 3 wt %.

Any of the couplers discussed above may also be used in the salt form, for instance sulphate, phosphate and hydrochloride, particularly sulphate or hydrochloride.

Compounds containing free amine groups are preferably used in the form of their salt. The salt form of such a compound forms a powder and is often more stable than the free base form.

The developer is an amino aromatic compound capable of being oxidised and undergoing a single electrophilic attack in the oxidised state. For instance it may be an aromatic system containing a single primary amine substituent.

The developer is such that it reacts substantially at only one position (normally the amine position). In some cases the structure of the developer may be such that it is possible that it reacts with other developer molecules, but it reacts preferentially with coupler molecules. Preferably the structure of the developer is such that it undergoes substantially no reaction with other developer molecules.

Suitable developers include o-nitro and p-nitro α-naphthylamines of the formulae

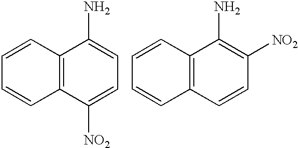

Other suitable developers include o- and p-nitrophenylamines H₂N—Ph—NO₂, N,N-disubstituted o-phenylene diamines and N,N-disubstituted p-phenylene diamines.

The developer can be an N,N-disubstituted p-phenylene diamine. These developers have an amine group protected by disubstitution and react only at the primary amine group. In this case it normally has the formula XIV, as follows:

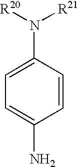

in which R²⁰ and R²¹ are each independently H, alkyl, cycloalkyl, alkenyl, cycloalkenyl, aryl, alkaryl, aralkyl, —R′NHCOR, —R′CONHR, —R′OH, —R′SO₂R, —R′SO₂NHR OR —R′OR in which R is alkyl, cycloalkyl, alkenyl, cycloalkenyl, aryl, alkaryl, aralkyl, and R′ is alkylene, cycloalkylene, alkenylene, cycloalkenylene, arylene, alkarylene or aralkylene, or substituted versions of any of these. Suitable substituting groups include OH, —OR, Cl, Br, F, —CO₂H, —CO₂R, —OR and —COR. Alternatively, R²⁰ and R²¹ may together form a substituted or unsubstituted cycloalkyl, cycloalkenyl or aryl ring.

Preferably R²⁰ and R²¹ are, independently, C₁₋₄ alkyl, preferably —CH₃, —CH₂CH₃ or i-propyl; C₁₋₃ hydroxyalkyl, preferably —CH₂CH₂OH; alkylene alkoxy, preferably ethylmethoxy (—CH₂CH₂OCH₂); or R 22SO₂NHR²² or R²²NHSO₂R²² in which R²² is C₁₋₃ alkyl, for instance —CH₂CH₂SO₂NHCH₃ or —CH₂CH₂NHSO₂CH₃.

Particularly preferred developers of the above formula XIV are those in which R²⁰ and R²¹ are both —CH₂CH₃ or R²³ is —CH₂CH₃ and R²¹ is —CH₂CH₂NHSO₂CH₃. The latter R²¹ substituent is believed to contribute to dermatological compatibility. In other suitable developers R²⁰ is ethyl and R²¹ is hydroxyethyl; or R²⁰ is ethyl and R²¹ is —CH₂CH₂CH₃; or R²⁰ is selected from H, methyl, ethyl, and propyl and R²¹ is selected from methyl, ethyl and propyl.

In general the developer may be selected from compounds having the general formula XV:

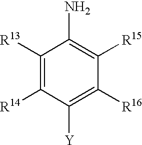

The group Y is a blocking group which ensures that reaction (under the conditions of dyeing the hair) takes place only at the primary amine group. The group Y for instance can be —NR²⁰R²¹ (as in formula XIV above). Other suitable Y groups include —NO₂, —CO₂H, —CO₂R, —COR and OH. R is as defined above for formula XIV.

In an alternative developer formula XVI, the blocking group Y is in the ortho-position relative to the amino group, giving the following formula XVI.

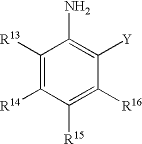

Thus the group Y is positioned so that the developer undergoes only one reaction, at the primary amine group, under the conditions of the reaction.

R¹³, R¹⁴, R¹⁵ and R¹⁶ can each be, independently, any of the groups listed for R¹ to R⁴ above. R¹³ and R¹⁴ together, and/or R¹⁵ and R¹⁶ together, may form a substituted or unsubstituted cycloalkyl, cycloalkenyl or aryl ring.

Preferably R¹³ to R¹⁶ are each, independently, H, methyl, ethyl, n-propyl, i-propyl, F, Cl, OH, NO₂, —CO₂H, —CO₂R or —COR.

In any of the above formulae the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

A preferred developer has the following formula XVII, as follows:

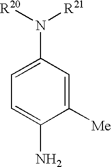

Specific examples of developers of the invention are those of the following structural formulae:

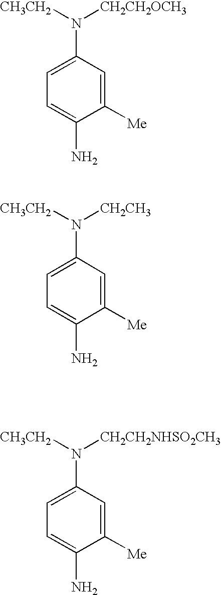

These are suitable especially f or colouring of damaged hair.

Additional developers of the invention are:

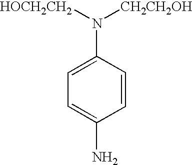

which tends to fade more rapidly than certain others, and

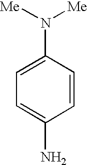

which is highly reactive.

Examples of further preferred developers, which are particularly suitable for coloration of undamaged hair, are 2,6-dichloro-p-aminophenol, 2-chloro-p-aminophenol, 3-chloro-p-aminophenol, 2,3-dichloro-p-aminophenol and 3,5-methyl-p-aminophenol.

The derivatives above include salts, for instance sulphate, phosphate and hydrochloride, particularly sulphate or hydrochloride. Salts are normally formed with the amine groups. The preferred developer in which R²⁰ is —CH₂CH₃ and R²¹ is CH₂CH₂NHSO₂CH₃ is often provided in salt form as a stable powder (more stable than the free base form). We have found that this salt forms such that it contains 3 moles of salt molecule to 2 moles of the free base molecule. A further useful salt is the hydrochloride salt of developers such as 2,6-dichloro-p-aminophenol.

Developer is often included in the composition in amounts of from 0.01 to 5 or 7% by weight based on total composition applied to the hair. Preferred amounts of developer are from 0.3 to 2 or 4%, preferably 0.4 to 1.5 or 3%.

For both developers and couplers the solubility properties can be important. The developer and coupler compounds themselves should have solubility such that they can be formulated in appropriate concentrations. For application at high pH they preferably have solubility of at least log, more preferably at least 15g and most preferably at least 20g/100 ml deionised water at pH about 10 and 25° C. They may have solubility at least 25g/100 ml, and even up to 50 or 80 g/100 ml but normally not more than 30g/100 ml.

The developer and coupler compounds are also generally such that the solubility of the final coloured dimer (or trimer if produced) is low under normal hair conditions and, especially, conditions of washing. Thus solubility (at pH about 8) of the final coloured molecule is preferably below 5 g/100 ml deionised water at 25° C., in particular below 2 or 1 g/100 ml and most preferably below 0.5 g/100 ml or even below 0.2 g/100 ml.

We find that if the developer and coupler compounds are sufficiently soluble in the composition, under the conditions of application to the hair, they will diffuse sufficiently rapidly into the hair shaft. However, the coloured molecules produced should be of sufficiently low solubility that they resist washing out of the hair. In compositions which are to be applied at high pH (for instance above pH 10), an indication of solubility can sometimes be given by pKa. Thus if one or more of the developers and couplers, in particular the developers, has an ionisable group which is substantially ionised at a pH of above 9, preferably above pH 10, this is an indicator of solubility at about pH 10. However, in the final coloured molecule and at the pH in the hair shaft (which is usually about pH 5.5 to 6) it becomes non-ionised. This gives an indication that under normal conditions it has reduced solubility. This can often be achieved by providing at least one group which has a pKa of from 8 to 12 (and is thus ionised above that pH) in a developer or coupler molecule and which on reaction to form a final coloured molecule also has pKa of from 8 to 12 (and is thus non-ionised at below that pH). Solubility can be affected by various factors but pKa can be a good indicator of likely solubility in some cases.

We find that an advantage of the colouring compounds of the invention is that they can give even coloration and fade resistance on both damaged and undamaged hair. This is particularly useful in cases where the hair has been dyed once and then allowed to grow so that undyed, undamaged hair appears. On redyeing, the undamaged hair and the faded, dyed, damaged hair must both be coloured and show even fade resistance. It is particularly important to be able to provide colour, wash fastness and fade resistance to damaged (eg bleached and/or permed and/or previously dyed) hair.

An advantage of the system of the invention is that the full range of colours can be achieved using a very small number of compounds, in contrast to standard oxidative dyeing systems. Preferably only one or two, in particular only one, developer compound is used. In particular it is preferred that this is used in combination with not more than three, or even only one or two compounds of each of the types (A), (B) and (C) discussed above.

All of these couplers and developers can be classed as “oxidative” colouring agents, since they require the presence of an oxidising agent to initiate their reaction. Preferably less than 0.1 wt %, more preferably less than 0.08 wt % or 0.05 wt % and in particular substantially no oxidative colouring agents are included which are not of types (i), (A), (B) and (C) or of the formulae X to XIII.

In particular it is preferred that no non-oxidative dyes are present and indeed preferably less than 0.1 wt %, especially 0.08 or 0.05 wt %, in particular substantially no other colouring components are included than the developer as defined and couplers (A), (B) and (C) and couplers of formulae XI to XIII. That is, in the hair colouring composition the colouring components consist essentially of developer (i), couplers (A), (B) and (C) and optionally couplers of formulae XI to XIII. Trivial amounts of other colouring components can of course be included provided they do not significantly influence the final colour.

It is preferred that the composition contains less than 0.1 wt %, especially less than 0.08 wt % and in particular less than 0.05 wt % and even substantially no oxidative dye materials which are capable of undergoing reaction more than once, under the oxidising conditions of the hair colouring reaction.

A preferred composition comprises not more than 0.1 wt % of any oxidative colouring agent which can react with itself under the conditions of hair colouring. Preferably it comprises not more than 0.08 wt % or 0.05 wt % of any such agent. More preferably the total amount of such agents does not exceed these values.

Usually the colouring compositions of the invention have pH above 6.1 or 6.5, preferably above 7, in particular above pH 8 or 9. A pH of from 9 to 12 is often suitable. The systems of the invention can also be incorporated into low pH (eg. pH 1 to 6) hair colouring systems described in our copending application number GB 9626713.3.

The composition of the invention may also include an anti-oxidant, as described in our copending application number 9710754.4.

For the developers and couplers to be effective in forming colour they require the presence of an oxidising agent. This oxidising agent is normally included in the composition just before it is applied to the hair. Normally the composition of the invention will be supplied in at least two individual packages such as bottles, the oxidising agent being included in one package and the developers and couplers being included in another.

A preferred oxidising agent is hydrogen peroxide. Other oxidising agents which may be used include other inorganic peroxygen oxidising agents, preformed organic peroxyacid oxidising agents and other organic peroxides such as urea peroxide, melamine peroxide, and mixtures of any of these.

Suitable oxidising agents are preferably water-soluble, that is they have a solubility of at least about log in 1,000 ml of deionised water at 25° C. (“Chemistry” C. E. Mortimer, 5th Edition, page 277).

Suitable inorganic alkali metal peroxides other than hydrogen peroxide include sodium periodate, sodium perbromate and sodium peroxide, and inorganic perhydrate salt oxidising compounds such as the alkali metal salts of perborates, percarbonates, perphosphates, persilicates, and persulphates. Inorganic perhydrate salts may be incorporated as monohydrates, tetrahydrates etc. Mixtures of two or more of such inorganic peroxygen oxidising agents can be used if desired. Alkali metal bromates and iodates are suitable, bromates being preferred.

Amounts of inorganic peroxygen oxidising agent which can be used in the composition are normally from 0.0003 mol 0.2 mol per 100 g of composition, preferably up to 0.1 mol/100 g.

Suitable preformed organic peroxyacid oxidising agents have the general formula R¹³C(O)OOH, in which R¹³ is selected from saturated or unsaturated, substituted or unsubstituted, straight or branched chain, alkyl, aryl or alkaryl groups with from 1 to 14 carbon atoms.

One class of organic peroxyacid compounds suitable for use in the invention is that of the amide substituted compounds of the following general formulae XX and XXI:

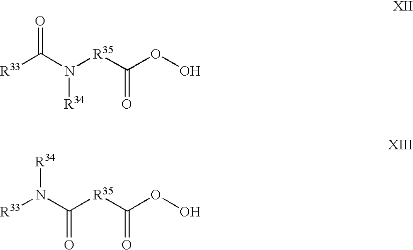

wherein R³³ is a saturated or unsaturated alkyl or alkaryl group or an aryl group, having from 1 to 14 carbon atoms, R³⁵ is a saturated or unsaturated alkyl or alkaryl group, or an aryl group, having from 1 to 14 carbon atoms and R³⁴ is H or a saturated or an unsaturated alkyl or alkaryl group, or an aryl group, having from 1 to 10 carbon atoms. Amide substituted organic peroxyacid compounds of this type are described in EP-A-170,386.

Other suitable organic peroxyacid oxidising agents include peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-phthaloylamino peroxycaproic, monoper succinic, nonanoyloxybenzoic, dodecanedioyl-monoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof. Mono- and diperazelaic acid, mono- and diperbrassylic acid and N-phthaloylaminoperoxicaproic acid and derivatives thereof are also suitable for use in the invention.

Preferred peroxyacid materials are selected from peracetic and pernanoic acids and mixtures thereof. Suitable amounts of preformed organic peroxyacid oxidising agents are from about 0.0001 to 0.1 mol per 100 g of compositions, preferably from about 0.001 to 0.05 mol, more preferably from about 0.003 to 0.04 mol, especially from about 0.004 to 0.03 mol/100 g.

The preformed organic peroxyacid oxidising agent, where present, is preferably present at a level of from about 0.01% to about 8%, more preferably from about 0.1% to about 6%, most preferably from about 0.2% to about 4%, and especially from about 0.3% to about 3% by weight of the hair colouring composition. The weight ratio of the inorganic peroxygen oxidising agent to the preformed organic peroxy acid is preferably in the range of from about 0.0125:1 to about 500:1, more preferably from about 0.0125:1 to about 50:1.

If additional organic peroxides are used, suitable amounts are from about 0.01% to about 3%, preferably from about 0.01% to about 2%, more preferably from about 0.1% to about 1.5% and most preferably from about 0.2% to about 1% by weight of composition.

An advantage of the systems of the invention is that very low levels of oxidising agent can be used if desired. Such systems are described in more detail in our copending application 9710756.9.

The composition may comprise ammonia, for instance in an amount of at least 0.01 wt %, preferably at least 0.05 wt % or 0.1 wt %.

In practice the composition of the invention may be supplied to the consumer as a single package containing developer and couplers. It is also possible to supply the composition so that the developers are individually packaged and the couplers are individually packaged. Couplers may be supplied as a preformed mixture selected to give a particular colour. Alternatively they can be supplied separately for mixing by the consumer to give a variety of different hair colours.

In all cases, the developers and couplers are mixed to form the composition of the invention before application to the hair. Generally oxidising agent, where supplied together with the colouring agents, is individually packaged separately from any of the colouring components. It is often mixed with the colouring components to form a component of the hair colouring composition before application to the hair. Alternatively it can be applied to the hair separately either before or after the hair colouring composition.

The developers, couplers and oxidising agent, and any other materials to be applied to the hair as components of the composition of the invention, may be provided in any suitable physical form. A preferred physical form is liquid. The liquid may be of low viscosity, for instance it may be water-thin, or it may be of higher viscosity. The material may be suspended in a gel network. The gel may be solid or of low viscosity.

The materials for colouring the hair are often formulated so that when they are mixed to form the composition of the invention for application to the hair they form a product of cream-like consistency, which is convenient for application to the hair. The final composition which is applied to the hair is often in the form of an emulsion.

Each individual material may be supplied in a form such that the composition containing it has a pH of above or below 7. For instance it may be from pH 1 to 11. In order to assist solubility of the various components, particularly developers and couplers, in a water-based carrier, the carrier may have a pH of above 7, for instance from pH 8 or 9 to pH 10 or 11. A pH as supplied of from 1 to 6 can assist in improving stability of the components.

The materials may be provided such that the pH of the final composition when mixed for application to the hair has a pH below 7 even though one of the components used to form it has a pH of above 7. Alcohols such as ethanol in amounts of from for instance 5 to 10 or 25% may be included to aid solubility of the developers and, particularly, the couplers in a water-based carrier.

In a second aspect of the invention we provide a hair colouring kit comprising as hair colouring components

(i) one or more developers as defined above, and

(ii) one or more couplers selected from (A), (B) and (C) as defined above, provided that at least one coupler (B) and/or (C) is present.

In this hair colouring kit any of the components may have any of the features discussed above for the composition of the invention.

In a third aspect of the invention we provide a method of colouring hair comprising providing

(i) one or more developers as defined above,

(ii) one or more couplers selected from (A), (B) and (c) as defined above, provided that at least one coupler (B) and/or (C) is present, and

(iii) an oxidising agent

and applying (i), (ii) and (iii) to the hair to be coloured.

In the method of the invention the components (i), (ii) and (iii) are preferably applied substantially simultaneously, particularly preferably mixed to form a single composition and then applied to the hair together. Within the term “substantially simultaneously” we also include application of one or more components to the hair followed by subsequent application of the remaining components within a period of not more than five minutes. The components may alternatively be applied to the hair non-simultaneously. For instance, the developer and couplers may be mixed and applied to the hair, followed or preceded by application of oxidising agent. Alternatively the oxidising agent may be applied simultaneously with either the developer or couplers, the developer and couplers being applied at different times.

The conditions of the reaction are normally those conventionally applied for dyeing hair. The temperature is normally from 10 to 45° C., often 20 to 35° C. pH can be low (eg below 7 or 6) but is often high, for instance above 6.5 or 7, or above 8 or 9 or even above 10.

In this specification, when leaving groups are discussed, as well as compounds which react only at one position or only with certain other compounds, we mean reaction under the conditions under which the colouring compounds will be applied to the hair.

In a fourth aspect of the invention we provide a hair colouring system which comprises

(i) one or more developers as defined above

(ii) one or more couplers selected from (A), (B) and (C) as defined above, provided that at least one coupler (B) or (C) is present, and optionally compounds of formulae X to XIII above,

which comprises no other oxidative colouring agents and the system is capable of providing a wide spectrum of colour shades without the use of additional colouring agents.

In the system of the fourth aspect of the invention, amounts and types of developer and coupler are selected so as to obtain the particular colour desired for any one application. For any different colour, selection is made from the same set of developers and couplers to provide that colour. Preferably the system can provide at least one light brown shade, at least one red shade and at least one dark brown shade. More preferably it also provides at least one blonde shade and at least one black shade.

Preferably the system comprises not more than three, preferably only one or two, compounds of each of the types (i), (A), (B) and (C). Thus in this preferred embodiment of the system of the invention a wide range of colours is obtained from as few as four colouring components.

The system may comprise, as couplers, couplers (A) and (B), optionally together with couplers of the formula

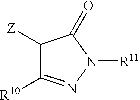

in which R¹⁰ and R¹¹ are as defined above, for instance alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR.

Preferably R¹⁰ is alkyl, alkenyl, aryl, alkaryl, aralkyl, R′NHCOR or R′CONHR, and R¹¹ is alkyl, alkenyl, aryl, alkaryl or aralkyl.

The system may also comprise instructions for selection of amounts and types of components (i) and (ii) to achieve a range of colours.

The system may be supplied to consumers, for instance those who wish to colour their own hair or to hairdressing salons, together with instructions to select particular amounts of each of the colouring components to obtain different colours. The system may also be used by manufacturers of hair colouring compositions. The manufacturer provides the colouring components of the four different types of and selects the amounts and types necessary for each individual colour which is intended to be marketed.

In the system of the invention any of the materials may be used which have been discussed above in the context of the composition of the invention.

The invention also provides new hair colouring compositions containing N,N-dimethyl acetoacetamide and/or N,N-diethyl acetoacetamide and/or the compound of the formula

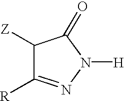

in which R is H, C₁₋₄ alkyl or phenyl.

These compositions may have any or all of the features discussed above.

Any of the compositions can contain various optional ingredients as follows.

Oxidative Dye Precursors

Preferably the only oxidative dye materials in the composition are materials (i) and (ii) discussed above. However, the compositions may optionally contain minor amounts of other oxidative dye materials. These may include those described in our copending application PCT/US97/22719, filed Dec. 9, 1997.

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear coloured. For example, oxidative primary intermediates capable of forming coloured polymers include materials such as aniline, which has a single functional group and which, on oxidation, forms a series of conjugated imines and quinoid dimers, trimers, etc. ranging in colour from green to black. Compounds such as p-phenylenediamine, which has two functional groups, are capable of oxidative polymerization to yield higher molecular weight coloured materials having extended conjugated electron systems. A representative list of primary intermediates and secondary couplers suitable for use herein is found in Sagarin, “Cosmetic Science and Technology”,” Interscience, Special Ed. Vol. 2 pages 308 to 310.

Non-Oxidative and Other Dyes

The hair colouring compositions used in the present invention may, in addition to the essential oxidative hair colouring agents (i) and (ii) and optional oxidative dyes, optionally include non-oxidative and other dye materials. Optional non-oxidative and other dyes suitable for use in the hair colouring compositions and processes according to the present invention include both semi-permanent, temporary and other dyes. Non-oxidative dyes as defined herein include the so-called “direct action dyes”, metallic dyes, metal chelate dyes, fibre reactive dyes and other synthetic and natural dyes. Various types of non-oxidative dyes are detailed in: “Chemical and Physical Behaviour of Human Hair” 3rd Ed. by Clarence Robbins (pp250-259); “The Chemistry and Manufacture of Cosmetics”. Volume IV. 2nd Ed. Maison G. De Navarre at chapter 45 by G. S. Kass (pp841-920); “Cosmetics: Science and Technology”, 2nd Ed., Vol. II, Balsam Sagarin, Chapter 23 by F. E. Wall (pp 279-343); “The Science of Hair Care” edited by C. Zviak, Chapter 7 pp 235-261 and “Hair Dyes”, J. C. Johnson, Noyes Data Corp., Park Ridge, U.S.A. (1973), (pp 3-91 and 113-139).

Direct action dyes, which do not require an oxidative effect in order to develop the color, are also designated hair tints and have long been known in the art. They are usually applied to the hair in a base matrix which includes surf actant material. Direct action dyes include nitro dyes such as the derivatives of nitroamino benzene or nitroaminophenol; disperse dyes such as nitroaryl amines, aminoanthraquinones or azo dyes; anthraquinone dyes, naphthoquinone dyes; basic dyes such as Acridine Orange C.I. 46005.

Nitro dyes are added to dyeing compositions to enhance colour of colorant and to add suitable aesthetic colour to the dye mixture prior to application.

Further examples of direct action dyes include the Arianor dyes basic brown 17, C.I.(color index)—no. 12,251; basic red 76, C.I.—12,245; basic brown 16, C.I.—12,250; basic yellow 57, C.I.—12,719 and basic blue 99, C.I.—56,059 and further direct action dyes such as acid yellow 1, C.I.—10,316 (D&C yellow no.7); acid yellow 9, C.I.—13,015; basic violet C.I.—45,170; disperse yellow 3, C.I.—11,855; basic yellow 57, C.I.—12,719; disperse yellow 1, C.I.—10,345; basic violet 1, C.I.—42,535, basic violet 3, C.I.—42,555; greenish blue, C.I.—42090 (FD&C Blue no.1); yellowish red, C.I.-14700 (FD&C red no.4); yellow, C.I.19140 (FD&C yellow no5); yellowish orange, C.I.15985 (FD&C yellow no.6); bluish green, C.I.42053 (FD&C green no.3); yellowish red, C.I.16035 (FD&C red no.40); bluish green, C.I.61570 (D&C green no.3); orange, C.I.45370 (D&C orange no.5); red, C.I.15850 (D&C red no.6); bluish red, C.I.15850 (D&C red no.7); slight bluish red, C.I.45380 (D&C red no.22); bluish red, C.I.45410 (D&C red no.28); bluish red, C.I.73360 (D&C red no.30); reddish purple, C.I.17200 (D&C red no.33); dirty blue red, C.I.15880 (D&C red no.34); bright yellow red, C.I.12085 (D&C red no.36); bright orange, C.I.15510 (D&C orange no.4); greenish yellow, C.I.47005 (D&C yellow no.10); bluish green, C.I.59040 (D&C green no.8); bluish violet, C.I.60730 (Ext. D&C violet no.2); greenish yellow, C.I.10316 (Ext. D&C yellow no.7);

Fibre reactive dyes include the Procion (RTM), Drimarene (RTM), Cibacron (RTM), Levafix (RTM) and Remazol (RTM) dyes available from ICI, Sandoz, Ciba-Geigy, Bayer and Hoechst respectively.

Natural dyes and vegetable dyes as defined herein include henna (Lawsonia alba), camomile (Matricaria chamiomila or Anthemis nobilis), indigo, logwood and walnut hull extract.

Temporary hair dyes, or hair coloring rinses, are generally comprised of dye molecules which are too large to diffuse into the hair shaft and which act on the exterior of the hair. They are usually applied via a leave-in procedure in which the dye solution is allowed to dry on the hair surface. As such these dyes are typically less resistant to the effects of washing and cleaning the hair with surface active agents and are washed off the hair with relative ease. Temporary hair dye can be used in the compositions of the invention and examples of preferred temporary hair dyes are illustrated below.

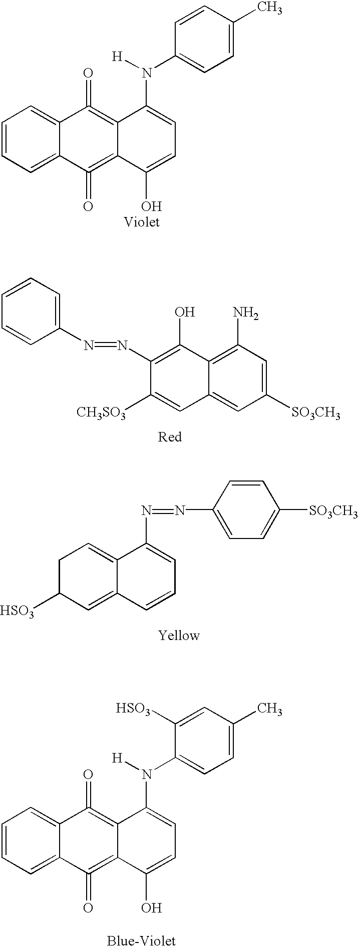

Semi-permanent hair dyes are dyes which are generally smaller in size and effect to temporary hair rinses but are generally larger than permanent (oxidative) dyes. Typically, semi-permanent dyes act in a similar manner to oxidative dyes in that they have the potential to diffuse into the hair shaft. However, semi-permanent dyes are generally smaller in size than the aforementioned conjugated oxidative dye molecules and as such are pre-disposed to gradual diffusion out of the hair again. Simple hair washing and cleaning action will encourage this process and in general semi-permanent dyes are largely washed out of the hair after about 5 to 8 washes. A semi-permanent dye system can be included in the compositions of the present invention. Suitable semi-permanent dyes for use in the compositions of the present invention are HC Blue 2, HC Yellow 4, HC Red 3, Disperse Violet 4, Disperse Black 9, HC Blue 7, HC Yellow 2, Disperse Blue 3, Disperse Violet 1 and mixtures thereof. Examples of semi-permanent dyes are illustrated below:

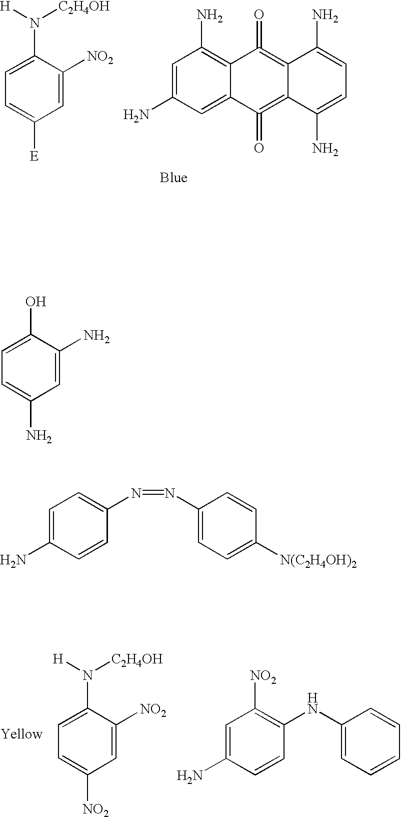

### Red

Typical semi-permanent dye systems incorporate mixtures of both large and small colour molecules. As the size of the hair is not uniform from root to tip the small molecules will diffuse both at the root and tip, but will not be retained within the tip, while the larger molecules will be generally only be able to diffuse into the ends of the hair. This combination of dye molecule size is used to help give consistent color results from the root to the tip of the hair both during the initial dyeing process and during subsequent washing.

Buffering Agents

If so desired, the compositions may contain one or more optional buffering agents and/or hair swelling agents (HSAs). Several different pH modifiers can be used to adjust the pH of the final composition or any constituent part thereof.

This pH adjustment can be effected by using well known acidifying agents in the field of treating keratinous fibres, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid, succinic acid and mixtures thereof. These are suitable for buffering to low pH.

Examples of alkaline buffering agents are ammonium hydroxide, ethylamine, dipropylamine, triethylamine and alkanediamines such as 1,3-diaminopropane, anhydrous alkaline alkanolamines such as mono or di-ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-arginine, lysine, alanine, leucine, iso-leucine, oxylysine and histidine and alkanolamines such as dimethylaminoethanol and aminoalkylpropanediol and mixtures thereof. Also suitable for use herein are compounds that form HCO₃ by dissociation in water (hereinafter referred to as “ion forming compounds”). Examples of suitable ion forming compounds are Na₂CO₃, NaHCO₃, K₂CO₃, (NH₄)₂CO₃, NH₄HCO₃, CaCO₃ and Ca(HCO₃) and mixtures thereof. These are suitable for buffering to high pH.

Preferred for use herein as buffering agents (to low pH) are organic and inorganic acids having a first pKa below pH 6, and their conjugate bases. As defined herein, first pKa means the negative logarithm (to the base 10) of the equilibrium constant, K, where K is the acid dissociation constant. Suitable organic and inorganic acids for use herein are: aspartic, maleic, tartaric, glutamic, glycolic, acetic, succinic, salicylic, formic, benzoic, malic, lactic, malonic, oxalic, citric, phosphoric acid and mixtures thereof. Particularly preferred are acetic, succinic, salicylic and phosphoric acids and mixtures thereof.

Catalyst

The colouring compositions herein may optionally contain a catalyst for any inorganic peroxygen oxidising agents and the optional preformed peroxy acid oxidising agent(s).

Thickeners

The colouring compositions of the present invention may additionally include a thickener at a level of from about 0.05% to about 20%, preferably from about 0.1% to about 10%, more preferably from about 0.5% to about 5% by weight. Thickening agents suitable for use in the compositions herein are selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM,) steareth-20 methacrylate copolymer; Aculyn 44 (RTM) polyurethane resin and Acusol 830 (RTM), acrylate copolymers which are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Diluent

Water is the preferred diluent for the compositions according to the present invention. However, the compositions according to the present invention may include one or more solvents as additional diluent materials. Generally, solvents suitable for use in the coloring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, 1,2-hexanediol, butoxyethanol, benzyl alcohol, and mixtures thereof. Water is the preferred principal diluent in the compositions according to the present invention. Principal diluent, as defined herein, means, that the level of that diluent present is higher than the total level of any other diluents.

The diluent is present at a level preferably of from about 5% to about 99.98%, preferably from about 15% to about 99.5%, more preferably at least from about 30% to about 99%, and especially from about 50% to about 98% by weight of the compositions herein.

Enzyme

A further additional material useful in the hair coloring compositions according to the present invention is one or more enzymes.

Suitable enzymatic materials include the commercially available lipases, cutinases, amylases, neutral and alkaline proteases, esterases, cellulases, pectinases, lactases and peroxidases conventionally incorporated into detergent compositions. Suitable enzymes are discussed in U.S. Pat. Nos. 3,519,570 and 3,533,139.

Peroxidases are haemoproteins specific for peroxide, but using a wide range of substances as donors. Catalase which decomposes peroxide, is included here in view of the fact that it is generally similar in structure and properties and is able to bring about certain oxidations by H₂O₂. The decomposition of H₂O₂ can be regarded as the oxidation of one molecule by the other. It is widespread in aerobic cells and may have some more important function. The coenzyme peroxidases are not haemoproteins and one at least is a flavoprotein. Other flavoproteins such as xanthine oxidase will also use H₂O₂ among other acceptors, and the coenzyme peroxidases resemble these rather than the classical peroxidases in not being specific for H₂O₂. Suitable peroxidases for the compositions of the present invention include horseradish peroxidase, Japanese radish peroxidase, cow's milk peroxidase, rat liver peroxidase, linginase and haloperoxidase such as chloro- and bromo-peroxidase.

Enzymes are optionally incorporated at levels sufficient to provide up to about 50 mg by weight, more typically about 0.01 mg to about 10 mg of active enzyme per gram of the hair treatment composition of the invention. Stated otherwise the peroxidase enzyme may be incorporated into the compositions in accordance with the invention at a level of from about 0.0001% to about 5%, preferably from about 0.001% to about 1%, more preferably from about 0.01% to about 1% active enzyme by weight of the composition. commercially available protease enzymes include those sold under the trade names Alcalase, Savinase, Primase, Durazym, and Esperase by Novo Industries A/S (Denmark), those sold under the tradename Maxatase, Maxacal and Maxapem by Gist-Brocades, those sold by Genencor International, and those sold under the tradename Opticlean and Optimase by Solvay Enzymes. Protease enzyme may be incorporated into the compositions in accordance with the invention at a level of from 0.0001% to 4% active enzyme by weight of the composition.

Amylases include, for example, α-amylases obtained from a special strain of B.licheniformis, described in more detail in GB-1,269,839 (Novo). Preferred commercially available amylases include for example, those sold under the tradename Rapidase by Gist-Brocades, and those sold under the tradename Termamyl and BAN by Novo Industries A/S. Amylase enzyme may be incorporated into the composition in accordance with the invention at a level of from 0.0001% to 2% active enzyme by weight of the composition.

Lipolytic enzyme may be present at levels of active lipolytic enzyme of from 0.0001% to 2% by weight, preferably 0.001% to 1% by weight, most preferably from 0.001% to 0.5% by weight of the compositions.

The lipase may be fungal or bacterial in origin being obtained, for example, from a lipase producing strain of Humicola sp., Thermomyces sp. or Pseudomonas sp. including Pseudomonas pseudoalcaligenes or Pseudomonas fluorescens. Lipase from chemically or genetically modified mutants of these strains are also useful herein. A preferred lipase is derived from Pseudomonas pseudoalcaligenes, which is described in Granted European Patent, EP-B-0218272.

Another preferred lipase herein is obtained by cloning the gene from Humicola lanuginosa and expressing the gene in Asperaillus oryza, as host, as described in European Patent Application, EP-A-0258 068, which is commercially available from Novo Industri A/S, Bagsvaerd, Denmark, under the trade name Lipolase. This lipase is also described in U.S. Pat. No. 4,810,414, Huge-Jensen et al, issued Mar. 7, 1989.

Surfactant Materials

The compositions of the present invention can additionally contain a surfactant system. Suitable surfactants for inclusion in the compositions of the invention generally have a lipophilic chain length of from about 8 to about 22 carbon atoms and can be selected from anionic, cationic, nonionic, amphoteric, zwitterionic surfactants and mixtures thereof.

(i) Anionic Surfactants

Anionic surfactants suitable for inclusion in the compositions of the invention include alkyl sulphates, ethoxylated alkyl sulphates, alkyl glyceryl ether sulfonates, methyl acyl taurates, fatty acyl glycinates, N-acyl glutamates, acyl isethionates, alkyl sulfosuccinates, alkyl ethoxysulphosuccinates, alpha-sulfonated fatty acids, their salts and/or their esters, alkyl ethoxy carboxylates, alkyl phosphate esters, ethoxylated alkyl phosphate esters, alkyl sulphates, acyl sarcosinates, hydrotropes, such as alkyl xylene sulphonate and fatty acid/protein condensates, and mixtures thereof. Alkyl and/or acyl chain lengths for these surfactants are C₁₂-C₂₂, preferably C₁₂-C₁₈, more preferably C₁₂-C₁₄.

(ii) Nonionic Surfactants

The compositions of the invention can also comprise water-soluble nonionic surf actant(s). Surf actants of this class include C₁₂-C₁₄ fatty acid mono- and diethanolamides, sucrose polyester surfactants and polyhydroxy fatty acid amide surfactants having the general formula below.

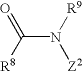

The preferred N-alkyl, N-alkoxy or N-aryloxy, polyhydroxy fatty acid amide surfactants according to the above formula are those in which R⁸ is C₅-C₃, hydrocarbyl, preferably C₆-C₁₉ hydrocarbyl, including straight-chain and branched chain alkyl and alkenyl, or mixtures thereof and R⁹ is typically hydrogen, C₁₋₈, alkyl or hydroxyalkyl, preferably methyl, or a group of formula —R¹—O—R² wherein R¹ is C₂-C₈ hydrocarbyl including straight-chain, branched-chain and cyclic (including aryl), and is preferably C₂-C₄ alkylene, R² is C₁-C₈ straight-chain, branched-chain and cyclic hydrocarbyl including aryl and oxyhydrocarbyl, and is preferably C₁-C₄ alkyl, especially methyl, or phenyl. Z² is a polyhydroxyhydrocarbyl moiety having a linear hydrocarbyl chain with at least 2 hydroxyls (in the case of glyceraldehyde) or at least 3 hydroxyls (in the case of other reducing sugars) directly connected to the chain, or an alkoxylated derivative (preferably ethoxylated or propoxylated) thereof. Z² preferably will be derived from a reducing sugar in a reductive amination reaction, and most preferably Z² is a glycityl moiety. Suitable reducing sugars include glucose, fructose, maltose, lactose, galactose, mannose, and xylose, as well as glyceraldehyde. As raw materials, high dextrose corn syrup, high fructose corn syrup, and high maltose corn syrup can be utilised as well as the individual sugars listed above. These corn syrups may yield a mix of sugar components for Z². It should be understood that it is by no means intended to exclude other suitable raw materials. Z² preferably will be selected from the group consisting of —CH₂—(CHOH)ₙ—CH₂OH, —CH(CH₂OH)—(CHOH)ₙ₋₁—CH₂H, CH₂(CHOH)₂(CHOR′)CHOH)—CH₂OH, where n is an integer from 1 to 5, inclusive, and R′ is H or a cyclic mono- or polysaccharide, and alkoxylated derivatives thereof. As noted, most preferred are glycityls wherein n is 4, particularly —CH₂—(CHOH)₄—CH₂OH.

The most preferred polyhydroxy fatty acid amide has the formula R⁸(CO)N(CH₃)CH₂(CHOH)₄CH₂OH wherein R⁸ is a C6-C19 straight chain alkyl or alkenyl group. In compounds of the above formula, ^{R8}—CO—N< can be, for example, cocoamide, stearamide, oleamide, lauramide, myristamide, capricamide, palmiamide, tallowamide, etc.

Suitable oil-derived nonionic surfactants for use herein include water soluble vegetable and animal-derived emollients such as triglycerides with a polyethyleneglycol chain inserted; ethoxylated mono- and diglycerides, polyethoxylated lanolins and ethoxylated butter derivatives. One preferred class of oil-derived nonionic surfactants for use herein have the general formula below;

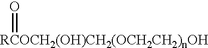

wherein n is from about 5 to about 200, preferably from about 20 to about 100, more preferably from about 30 to about 85, and wherein R comprises an aliphatic radical having on average from about 5 to 20 carbon atoms, preferably from about 7 to 18 carbon atoms.

Suitable ethoxylated oils and fats of this class include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, preferably glyceryl tallowate and glyceryl cocoate.

Preferred for use herein are polyethyleneglycol based polyethoxylated C₉-C₁₅ fatty alcohol nonionic surfactants containing an average of from about 5 to about 50 ethyleneoxy moieties per mole of surfactant.

Suitable polyethylene glycol based polyethoxylated C₉-C₁₅ fatty alcohols suitable for use herein include C₉-C₁₁ Pareth-3, C₉-C₁₁, Pareth-4, C₉-C₁₁, Pareth-5, C₉-C₁₁, Pareth-6, C₉-C₁₁ Pareth-7, C₉-C₁₁, Pareth-8, C₁₁-C₁₅ Pareth-3, C₁₁-C₁₅ Pareth-4, C₁₁-C₁₅ Pareth-5, C₁₁-C₁₅ Pareth-6, C₁₁-C₁₅ Pareth-7, C₁₁-C₁₅ Pareth-8, C₁₁-C₁₅ Pareth-9, C₁₁-C₁₅ Pareth-10, C₁₁-C₁₅ Pareth-11, C₁₁-C₁₅ Pareth-12, C₁₁-C₁₅ Pareth-13 and C₁₁-C₁₅ Pareth-14. PEG 40 hydrogenated castor oil is commercially available under the tradename Cremophor (RTM) from BASF. PEG 7 glyceryl cocoate and PEG 20 glyceryl laurate are commercially available from Henkel under the tradenames Cetiol (RTM) HE and Lamacit (RTM) GML 20 respectively. C₉-C₁₁, Pareth-8 is commercially available from Shell Ltd under the tradename Dobanol (RTM) 91-8. Particularly preferred for use herein are polyethylene glycol ethers of ceteryl alcohol such as Ceteareth 25 which is available from BASF under the trade name Cremaphor A25.

Nonoxynol surfactants may also be used.

Also suitable for use herein are nonionic surfactants derived from composite vegetable fats extracted from the fruit of the Shea Tree (Butyrospermum Karkii Kotschy) and derivatives thereof. Similarly, ethoxylated derivatives of Mango, Cocoa and Illipe butter may be used in compositions according to the invention. Although these are classified as ethoxylated nonionic surfactants it is understood that a certain proportion may remain as non-ethoxylated vegetable oil or fat.

Other suitable oil-derived nonionic surfactants include ethoxylated derivatives of almond oil, peanut oil, rice bran oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil.

(iii) Amphoteric Surfactants

Amphoteric surfactants suitable for use in the compositions of the invention include:

(a) imidazolinium surfactants of the formula (1)

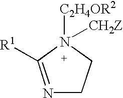

wherein R¹ is C₇-C₂₂ alkyl or alkenyl, R² is hydrogen or CH₂Z, each Z is independently CO₂M or CH₂CO₂M, and M is H, alkali metal, alkaline earth metal, ammonium or alkanolammonium; and/or ammonium derivatives of the formula (2)

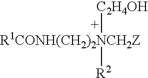

wherein R¹, R² and Z are as defined above;

(b) aminoalkanoates of the formula (3)

R¹NH(CH₂)ₙCO₂M

iminodialkanoates of the formula (4)

R′N[(CH₂)ₘCO₂M]₂

and iminopolyalkanoates of the formula (5)

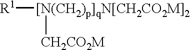

wherein n, m, p, and q are numbers from 1 to 4, and R¹ and M are independently selected from the groups specified above; and

(c) mixtures thereof.

Suitable amphoteric surfactants of type (a) are marketed under the trade name Miranol and Empigen and are understood to comprise a complex mixture of species. In practice, a complex mixture of cyclic and non-cyclic species is likely to exist and both definitions are given here for sake of completeness. Preferred for use herein, however, are the non-cyclic species.

Examples of suitable amphoteric surfactants of type (a) include compounds of formula (1) and/or (2) in which R₁ is C₈H₁₇ (especially iso-capryl), C₉H₁₉ and C₁₁H₂₃ alkyl. Especially preferred are the compounds in which R¹ is CH₁₉, Z is CO₂M and R² is H; the compounds in which R¹ is C₁₁H₂₃, Z is CO₂M and R² is CH₂CO₂M; and the compounds in which R¹ is C₁₁H₂₃, Z is CO₂M and R² is H.

In CTFA nomenclature, materials suitable for use in the present invention include cocoamphocarboxypropionate, cocoamphocarboxy propionic acid, and especially cocoamphoacetate and cocoamphodiacetate (otherwise referred to as cocoamphocarboxyglycinate). Specific commercial products include those sold under the trade names of Ampholak 7TX (sodium carboxy methyl tallow polypropyl amine), Empigen CDL60 and CDR 60 (Albright & Wilson), Miranol H2M Conc. Miranol C2M Conc. N.P., Miranol C2M Conc. O.P., Miranol C2M SF, Miranol CM Special (Rhone-Poulenc); Alkateric 2CIB (Alkaril Chemicals); Amphoterge W-2 (Lonza, Inc.); Monateric CDX-38, Monateric CSH-32 (Mona Industries); Rewoteric AM-2C (Rewo Chemical Group); and Schercotic MS-2 (Scher Chemicals). Further examples of amphoteric surfactants suitable for use herein include Octoxynol-1 (RTM), polyoxethylene (1) octylphenyl ether; Nonoxynol-4 (RTM), polyoxyethylene (4) nonylphenyl ether and Nonoxynol-9, polyoxyethylene (9) nonylphenyl ether.

It will be understood that a number of commercially-available amphoteric surfactants of this type are manufactured and sold in the form of electroneutral complexes with, for example, hydroxide counterions or with anionic sulfate or sulfonate surf actants, especially those of the sulfated C₈-C₁₈ alcohol, C₈-C₁₈ ethoxylated alcohol or C₈-C₁₈ acyl glyceride types. Note also that the concentrations and weight ratios of the amphoteric surfactants are based herein on the uncomplexed forms of the surfactants, any anionic surfactant counterions being considered as part of the overall anionic surfactant component content.

Examples of preferred amphoteric surfactants of type (b) include N-alkyl polytrimethylene poly-, carboxymethylamines sold under the trade names Ampholak X07 and Ampholak 7CX by Berol Nobel and also salts, especially the triethanolammonium salts and salts of N-lauryl-beta-amino propionic acid and N-lauryl-imino-dipropionic acid. Such materials are sold under the trade name Deriphat by Henkel and Mirataine by Rhone-Poulenc.

(iv) Zwitterionic Surfactants

Water-soluble auxiliary zwitterionic surfactants suitable for inclusion in the compositions of the present invention include alkyl betaines of the formula R⁵R⁶R⁷N⁺(CH₂)ₙCO₂M and amido betaines of the formula (6) below:

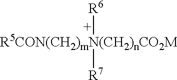

wherein R⁵ is C₁₁-C₂₂ alkyl or alkenyl, R⁶ and R⁷ are independently C₁-C₃ alkyl, M is H, alkali metal, alkaline earth metal, ammonium or alkanolammonium, and n, m are each numbers from 1 to 4. Preferred betaines include cocoamidopropyldimethylcarboxymethyl betaine, laurylamidopropyldimethylcarboxymethyl betaine and Tego betaine (RTM).

Water-soluble auxiliary sultaine surfactants suitable for inclusion in the compositions of the present invention include alkyl sultaines of the formula (7) below:

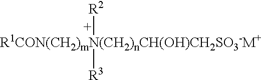

wherein R¹ is C₇ to C₂₂ alkyl or alkenyl, R² and R³ are independently C₁ to C₃ alkyl, M is H, alkali metal, alkaline earth metal, ammonium or alkanolammonium and m and n are numbers from 1 to 4. Preferred for use herein is coco amido propylhydroxy sultaine.

Water-soluble auxiliary amine oxide surfactants suitable for inclusion in the compositions of the present invention include alkyl amine oxide R⁵R⁶R⁷NO and amido amine oxides of the formula (8) below:

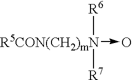

wherein R⁵ is C₁l to C₂₂ alkyl or alkenyl, R⁶ and R⁷ are independently C₁ to C₃ alkyl, M is H, alkali metal, alkaline earth metal, ammonium or alkanolammonium and m is a number from 1 to 4. Preferred amine oxides include cocoamidopropylamine oxide, lauryl dimethyl amine oxide and myristyl dimethyl amine oxide.

Additional Optional Materials

A number of additional optional materials can be added to the coloring compositions herein described, each at a level of from about 0.001% to about 5%, preferably from about 0.01% to about 3%, more preferably from about 0.05% to about 2% by weight of composition. Such materials include proteins and polypeptides and derivatives thereof; water-soluble or solubilizable preservatives; natural preservatives such as benzyl alcohol, potassium sorbate and bisabalol, benzoic acid, sodium benzoate and 2-phenoxyethanol; dye removers such as oxalic acid, sulphated castor oil, salicylic acid and sodium thiosulphate; H₂O₂ stabilisers; moisturising agents such as hyaluronic acid, chitin, and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663 as well as methyl cellulose, starch, higher fatty alcohols, paraffin oils, fatty acids and the like; solvents; anti-bacterial agents such as Oxeco (phenoxy isopropanol); low temperature phase modifiers such as ammonium ion sources (e.g. NH₄ Cl); viscosity control agents such as magnesium sulfate and other electrolytes; quaternary amine compounds such as distearyl-, dilauryl-, di-hydrogenated beef tallow-, dimethyl ammonium chloride, dicetyldiethyl ammoniumethylsulphate, ditallowdimethyl ammonium methylsulphate, disoya dimethyl ammonium chloride and dicoco dimethyl ammonium chloride; hair conditioning agents such as silicones, higher alcohols, cationic polymers and the like; enzyme stabilisers such as water soluble sources of calcium or borate species; colouring agents; TiO₂ and TiO₂-coated mica; perfumes and perfume solubilizers; and zeolites such as Valfour BV400 and derivatives thereof and Ca²⁺/Mg²⁺ sequestrants such as polycarboxylates, amino polycarboxylates, polyphosphonates, amino polyphosphonates etc. and water softening agents such as sodium citrate. Other optional materials include anti-dandruff actives such as ZPT, and perfumes.

### EXAMPLES

The invention will now be illustrated by the following examples. In these examples, various standard tests are used, as follows.

I Assessment of Initial Colour and Colour Change (Measurement of ΔE)

The equipment used to measure both the initial colour and colour change of substrates (hair/skin) dyed with the low pH colouring compositions of the present invention is a Hunter Colourquest spectrophotometer. The value used to express the degree of colour change on any particular substrate is Delta E (ΔE). Delta E, as defined herein, is represented by a factual sum of L, a, and b values such that:

ΔE=(ΔL²+Δa²+Δb²)^{½}

and L is measure of lightness and darkness (colour intensity), wherein L=100 is equivalent to white, and L=0 is equivalent to black. Further, “a” is a measure of the red and green quotients (colour hues) such that positive equates to red and negative to green and “b” is a measure of the yellow and blue quotients (colour hues) such that positive equates to yellow and negative equates to blue.

Hunter Colourquest measurements can be carried out on the Hunter Labscan Colourimeter which is a full scanning spectrocolorimeter with a wavelength of from 400-700 nanometers which records the colour of test hair switches (tresses) in terms of “L”, “a” and “b” values. The machine is set to: mode—0/45; port size—1 inch; view size—1 inch; light—D65; field of view—10°; UV lamp/filter—none. The hair is placed in a sample holder designed to hold the hair in uniform orientation during measurement. Equivalent calorimeters can be used, but it must be ensured that the hair does not move during measurement. The hair must be spread to cover the 1 inch port during colour measurement. Dots are placed on the switch holder to guide the positioning of the holder at the port. The dots are lined up with a mark on the port and readings are taken at each spot.

Eight measurements are run per switch, 4 on each side, and three switches are run per treatment.

II Standard Hair Switch

The compositions according to the present invention can be used to colour hair of all colours, types and condition. For the purposes of illustration various test hair switches can be tested herein. Two of these standard hair switches can be measured in terms of their approximate L, a, b values.

L a b

| Light brown | about 60 | about 9 | about 32 |
|---|---|---|---|
| (permed and bleached) | | | |
| 40% grey dark brown | 35-37 | 4.5—5.5 | 11.5-12.7 |

Yak hair (virgin or permed and/or bleached) can also be used. It has values of: L=about 82 to 83, a=about −0.5 to 0.7, b=about 11 to 12.

III Hair Switch Colouring Method

To colour hair, a 4 gramme switch of about 8 inch long hair (or a 2 gramme switch of 4 inch long hair) is hung over a suitable container. The test colouring product is then prepared (ie, where applicable the separate bottle components are mixed together) and about 2 grammes of product per gramme hair is applied directly to the test hair switch. The colourant is massaged through the hair switch for up to about 1 minute and then left on the hair switch for up to about 30 minutes. After rinsing with running water for about 1 or 2 minutes the coloured hair switch is then cleansed (according to the shampoo protocol IV below) and dried. Drying can be effected either naturally (without heat assistance) or using a drier. The colour development (initial colour) of the coloured, cleansed, dried test hair switch can then be assessed using the Hunter Colourquest spectrophotometer.

For the delivery of a red shade (hue) to prepermed, prebleached light brown hair (having L, a, b values of approximately 60, 9 and 32) the preferred initial shade of the coloured hair will have a hue value (arc tangent of (b/a)) in the range of from about 25 to about 70, more preferably from about 30 to about 65, most preferably from about 35 to about 60 and wherein the initial colour intensity (L) is greater than about 10 and less than about 70, preferably greater than about 15 and less than about 65 more preferably greater than about 20 and less than about 60.

For the delivery of a brown or black shade (hue) to prepermed, prebleached light brown hair (having L, a, b values of approximately 60, 9 and 32) the preferred initial shade of the coloured hair will have a hue value (arc tangent of (b/a)) of less than about 25, preferably less than about 20 and the initial colour intensity (L) will be greater than about 1 and less than about 50, preferably greater than about 5 and less than about 45.

For the delivery of a light brown shade (hue) to prepermed, prebleached light brown hair (having L, a, b values of approximately 60, 9 and 32) the preferred initial shade of the coloured hair will have a hue value (arc tangent of (b/a)) in the range of from about 70 up to about 110 and wherein the initial colour intensity (L) will be greater than about 20 and less than about 95, preferably greater than about 25 and less than about 90.

A significant colour change, as delivered via the colouring compositions according to the present invention often means a colour change on permed and bleached hair in terms of Delta E which is preferably greater than about 5 or 8, preferably greater than about 10, more preferably greater than about 12, most preferably greater than about 15 and especially greater than about 20.

IV Hair Switch Cleansing Method

Switches of coloured hair are subjected to a repeated cleansing cycle wherein the following process is repeated.

A 4 gramme, 8 inch test switch (or a 2 gramme, 4 inch test switch) of coloured hair is clamped over a suitable container and rinsed thoroughly for about 10 seconds using warm water (at about 100° F. at about 1.5 gallons/minute pressure). Shampoo (about 0.1 ml non-conditioning shampoo per gramme hair) can then be applied directly to the wet test switch using a syringe. After lathering the hair for about 30 seconds the hair is rinsed in running water for about 30 seconds. The shampoo and lathering process is then repeated with a final 60 second rinse. Excess water can be removed (squeezed) from the test switch using the fingers. The test switch is then dried either naturally, or using a pre-heated dryer box at about 140° F. (for about 30 minutes). The coloured, cleansed, dried test hair switch can then be colour assessed (Delta E fade).

During any single test cycle each different switch to be assessed should be tested in water at equivalent temperature, pressure level and hardness level.

Delta E fade results for prepermed, prebleached light brown hair (having L, a, b values approximately 60, 9 and 32) which has been coloured a red shade (of hue value in the range of from about 25 to about 70) are preferably less than about 5.0, preferably less than about 4.5, more preferably less than about 4.0, particularly preferably less than about 3.0 or 2.0 and wherein the change in hair colour, % delta E, after up to 20 washes, is less than about 20%, and preferably less than about 15%, more preferably less than about 10%.

Delta E fade results for prepermed, prebleached light brown hair (having L, a, b values of approximately 60, 9 and 32) which has been coloured a brown or black shade (of hue value of less than about 25) are preferably less than about 2.3, preferably less than about 2.0, more preferably less than about 1.7 and wherein the change in hair colour, % delta E, after up to 20 washes, is less than about 5%, preferably less than about 4.5%, more preferably less than about 4%, most preferably less than about 3.5%.

Delta E fade results for prepermed, prebleached light brown hair (having L, a, b values of approximately 60, 9 and 32) which has been coloured a light brown shade (of hue in the range of from about 70 to about 110) are preferably less than about 2.6, preferably less than about 2.3 and wherein the change in hair colour, % delta E, after up to 20 washes, is less than about 15%, preferably less than about 12%, more preferably less than about 10%, most preferably less than about 8%.

In preferred embodiments herein, the change in the colour of the dyed hair over time (Delta E fade) is less than about 15%, preferably less than about 12%, more preferably less than about 10% and most preferably less than about 8%.

V Perming Protocol

The following method is used to perm hair which is usually subsequently to be bleached.

A 4 gram switch of about 8 inch long hair is hung over a suitable container. Perming solution supplied under the trade name “Zotos” is applied to the hair so as to saturate it totally. The switches are then resaturated. The switches are then laid on a plastic tray for 20 minutes and subsequently rinsed for 1½ to 2 minutes with tap water at 37° C. The switches are squeezed dry and towelled dry. The switches are then hung over the container again and commercially available “Zotos” neutraliser is applied so as to saturate them. They are then laid in the plastic tray for 5 minutes and subsequently rinsed for 1½ to 2 minutes in tap water at 37° C. The switches are then shampooed twice and left to dry.

VI Bleaching Protocol

The prepermed switches are dried for 20 minutes and hung over the edge of the container. A maximum of 9 or 10 switches at once are treated. The commercially available bleach from Clairol, “Born Blonde (with chamomile)” is mixed according to the instructions and 10 grams of the material is applied to each switch and massaged in thoroughly. Each switch is wrapped loosely in clingfilm and left for 30 minutes. It is subsequently rinsed for 2 minutes in tap water at 37° C. It is then shampooed once.

### Example 1

In this example formulation 1 according to the invention is compared with a commercially available product and the two dyeing compositions compared for colour fading, measured by ΔE.

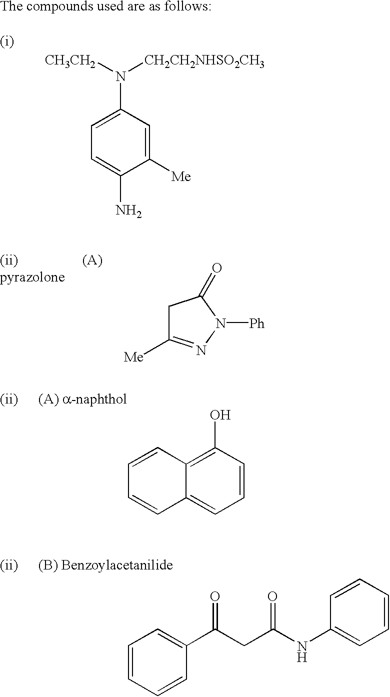

Formulation 1 had the following composition:

| | % by weight |
|---|---|
| Ceteareth 25 | 0.84 |
| Cetyl alcohol | 1.16 |
| Stearyl alcohol | 1.16 |
| (i) | 0.87 |
| Pyrazolone (i) (C) | 0.16 |
| α-naphthol (ii) (A) | 0.0046 |
| Benzoylacetaflilide (ii) (B) | 1.86 |
| Ethanol | 9.3 |
| NH₄OH | 1.6 |
| Hydrogen peroxide | 3.0 |
| Sodium Sulphite | 0.46 |
| EDTA | 0.46 |
| Water | up to 100 |

The pH of formulation 1 was from 9 to 10.

The comparative formulation was L'Oreal Recital “Santiago”.

8 grams of each formulation was applied to a 4 gram switch of light brown permed and bleached hair for 30 minutes as described in the colouring method above. Perming and bleaching were carried out according to the protocols described above.

Values of L, a and b were calculated on the undyed hair and the dyed hair. The difference in colour between the two states was calculated to give ΔE initial, in the manner described above for measuring ΔE.

The dyed switch was then subjected to 40 washes, using the washing protocol described above. After 40 washes the values of L, a and b were again measured and the difference in colour between the initial dyed switch and the washed switch was calculated as described above for ΔE, to give ΔE fade.

Values of ΔE initial, % fade and ΔE fade are given below.

| | | | | ΔE | ΔE | |
|---|---|---|---|---|---|---|
| Formula I | L | a | b | initial | fade | % fade |
| Before | 60.15 | 9.94 | 32.08 | | | |
| Dyed | 29.2 | 13.55 | 13.89 | 36.1 | | |
| 40 washes | 30.34 | 13.24 | 13.50 | | 1.25 | 3.5 |
| L'Oreal | | | | | | |
| Before | 58.95 | 9.68 | 31.63 | | | |
| Dyed | 30.06 | 13.66 | 14.68 | 33.8 | | |
| 40 washes | 36.69 | 8.76 | 18.21 | | 9.00 | 26.6 |

These results show that the commercial formulation and Formula I of the invention gave a similar ΔE initial, with the formulation of the invention in fact giving a greater ΔE and consequently a greater change in colour upon dyeing. However it will then be seen that the ΔE fade after 40 washes is significantly lower for formula 1 of the invention than for the commercially available product.

### Example 2

This example demonstrates how the system of the invention can give a broad colour range using just four compounds.

Three formulations were tested using the four materials (i), (ii) (A), (ii) (B) and (ii) (C) above as the only colouring agents. The formulations are numbered 2, 3 and 4 and are as follows:

| Components | Formulation 2 | Formulation 3 | Formulation 4 |
|---|---|---|---|
| Ceteareth 25 | 1.74 | 0.82 | 0.94 |
| Stearyl Alcohol | 2.6 | 1.24 | 1.4 |
| Cetyl Alcohol | 2.6 | 1.24 | 1.4 |
| (i) | 0.42 | 0.92 | 1.4 |
| (ii) (C | 0.031 | 0.16 | 0.13 |
| (ii) (A) | 0.007 | 0.0049 | 0.02 |
| (ii) (B) | 0.1 | 1.9 | 1.7 |
| Sodium Sulphite | 0.3 | 0.59 | 0.5 |
| SOTA | 0.3 | 0.59 | 0.5 |
| Ethanol | 6.25 | 9.9 | 21 |
| NH₄OH | 0.5 | 0.53 0.5 | |
| Hydrogen | 1 | 3 | 1 |
| Peroxide | | | |
| Water | Up to 100% | Up to 100% | Up to 100% |
| pH | 9-10 | 9-10 | 9-10 |

In the above compositions all components are given by weight of total composition. The light brown permed and bleached 4 gram switch of hair was coloured in the same way as described in Example 1. The initial colour change ΔE initial and the final colour are given below.

| Formula | L | a | b | ΔE initial | Colour |
|---|---|---|---|---|---|
| 1 | 34.3 | 6.2 | 17.9 | 29.0 | Light Brown |
| 2 | 26.4 | 15.9 | 10.5 | 35.8 | Auburn |
| 3 | 16.6 | 3.85 | 4.5 | 52.5 | Brown |

It can be seen that this set of only four colouring components is capable of giving a range which encompasses light brown, auburn and brown.

### Example 4

The comparisons-below demonstrate the advantages of a system according to the invention in comparison with a system disclosed in GB 1,025,916. The following formulations were tested

| Formula | I | II | III | IV |
|---|---|---|---|---|
| Cetyl alcohol | 1.4 | 1.4 | 1.4 | 1.4 |
| Stearyl alcohol | 1.4 | 1.4 | 1.4 | 1.4 |
| Ceteareth-25 | 1.0 | 1.0 | 1.0 | 1.0 |
| 4-(N-ethyl,N-hydroxyethyl-p- | 1.1 | 1.1 | 1.1 | 1.1 |
| phenylenediamifle | | | | |
| p-Carboxybenzoylacetanilide | 2% | 3.6% | / | / |
| N,N-Diethylacetoacetamide | / | / | 2% | 2% |
| EDTA | / | / | / | 0.2% |
| Ascorbic acid | / | / | / | 0.4% |
| Ammonia | 1.4 | 1.4 | 1.4 | 1.4 |
| Peroxide | 2.9 | 2.9 | 2.9 | 2.9 |
| Water | to 100 | to 100 | to 100 | to 100 |

Experimentation was carried out on either on virgin (undamaged) yak hair or permed (damaged) yak hair. All formulae were applied to the relevant switch at RT for 30 minutes at pH about 10. Formulae I and III contain the two different couplers in a 1:1 weight ratio and formulae II and IV contain the two different couplers in a molar ratio of 1:1. Formulation IV contains ascorbic acid as anti-oxidant. Results were as follows.

| | | | delta E | |
|---|---|---|---|---|
| | | delta E | fade 20 | |
| Switch | Formula | uptake | washes | Comments |
| virgin yak | I | 32 | 4.5 | light brown colour - poor |
| | | | | yellow uptake |
| virgin yak | II | 31 | 3.0 | poor yellow colour |
| virgin yak | III | 41 | 1.5 | light yellow |
| permed yak | I | 29 | 6.5 | light green yellow |
| permed yak | II | 28 | 5.5 | light green yellow |
| permed yak | IV | 48 | 2.5 | bright intense green yellow |

Formulations III and IV contain a coupler (B) according to the invention and formulations I and II contain a yellow coupler used in GB 1025916. It can be seen that the formulations according to the invention give the best uptake and fade values on each type of hair. Fade values are generally lower for the virgin yak hair than for the permed yak hair, since permed hair tends to be more porous and to fade more rapidly in general.

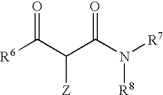

in which R⁶ is selected from the group consisting of methyl, ethyl, n-propyl, i-propyl, t-butyl and phenyl and R⁷ and R⁸ are independently selected from the group consisting of methyl, ethyl, n-propyl, hydroxymethyl and hydroxypropyl,

or (4) of the formula

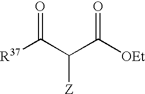

in which R³⁷ is C₄ alkyl,

and in all of which Z is an active leaving group, and

(C) compounds of the formula

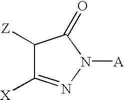

in which X is a non-leaving substituent and in which Z is an active leaving group and in which A is H or methyl,

such that in the presence of an oxidising agent the or each developer reacts with the or each coupler substantially only at the position having the active leaving group

and provided that the composition includes at least one coupler (B) or (C).

1. A hair colouring composition comprising
(i) one or more developers selected from amino aromatic systems capable of being oxidised and thereafter undergoing a single electrophilic attack, and
(ii) one or more couplers selected from
(A) (1) naphthols having an active leaving group in the Para-position relative to the OH group,
or (2) phenols of the formula 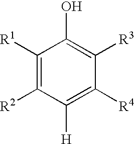
in which the H para to the OH group is an active leaving group and R¹, R², R³ and R⁴ are each independently selected from the group consisting of H, OH, methyl, ethyl, n-propyl, i-propyl, t-butyl, NH₂, CO₂H, CO₂R and COR, in which R is substituted or unsubstituted alkyl or alkenyl,
(B) 1,3-diketones (1) containing the group 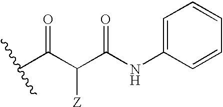
in which the N-phenyl group has no carboxy substituents,
or (2) containing the group 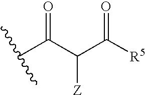
in which R⁵ contains an aryl group,
or (3) of the formula

2. A composition according to claim 1 in which the one or more developers (i) are selected from N,N-disubstituted phenylene diamines.

3. A composition according to claim 1 or claim 2 in which coupler (A) is selected from (1) naphthols having an active leaving group in the para-position relative to the OH group, and coupler (B) is selected from 1,3-diketones (1) containing the group 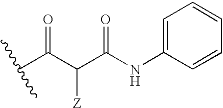
in which the N-phenyl group has no carboxy substituents,
or (2) of the formula 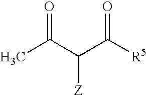
in which R⁵ contains an aryl group.

4. A composition according to any preceding claim in which coupler (B) is of the formula IV 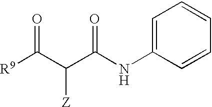
in which R⁹ is selected from alkyl, alkenyl, alkaryl, aryl and aralkyl, preferably phenyl and C₁₋₃ alkyl.

5. A composition according to any preceding claim in which at least one coupler (A) is α-naphthol.

6. A composition according to any preceding claim in which at least one coupler (B) is acetoacetanilide or benzoylacetanilide.

7. A composition according to any preceding claim in which the one or more developers (i) are selected from compounds of the formula 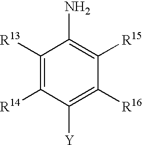
and compounds of the formula 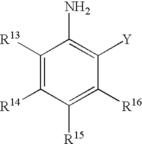
in which Y is selected from —NO₂, —CO₂H, —CO₂R, —COR and OH and R¹³, R¹⁴, R¹⁵ and R¹⁶ are independently selected from H, methyl, ethyl, n-propyl, i-propyl, F, Cl, OH, NO₂, —CO₂H, —CO₂R and —COR.

8. A composition according to any preceding claim in which at least one coupler (A) is 3-aminophenol.

9. A composition according to any preceding claim in which at least one coupler (B) is N,N-diethyl acetoacetamide or N,N-dimethyl acetoacetamide.

10. A composition according to any preceding claim in which at least one coupler (C) is selected from compounds of the formula 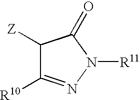
in which R¹⁰ is selected from the group consisting of H, methyl, and phenyl and R¹¹ is H or methyl.

11. A composition according to any preceding claim in which at least one developer (i) is selected from the group consisting of 2,6-dichloro-p-aminophenol, 2-chloro-p-aminophenol, 3-chloro-p-aminophenol, 2,3-dichloro-p-aminophenol and 3,5-methyl-p-aminophenol.

12. A composition according to any preceding claim which includes not more than two couplers of each of the types (A), (B) and (C).

13. A composition according to any preceding claim which includes only one developer compound (i).

14. A composition according to any preceding claim which contains not more than 0.1 wt % oxidative colouring agents which are not of the types (i), (A), (B) and (C) or of the formulae X to XIII given herein.

15. A composition according to any preceding claim which contains not more than 0.1 wt % oxidative colouring agents which are capable of undergoing reaction more than once under the conditions of hair colouring.

16. A composition according to any preceding claim which contains not more than 0.1 wt % of any oxidative colouring agent which can react with itself under the conditions of hair colouring.

17. A composition according to any preceding claim in which substantially all molecules of developer (i) do not react substantially with other developer molecules, under the conditions of hair colouring.

18. A composition according to any preceding claim which has a pH of at least 6.1, preferably at least 6.5.

19. A composition according to any preceding claim which contains ammonia or ammonium hydroxide in an amount of at least 0.01 wt %.

20. A hair colouring kit comprising as hair colouring components
(i) one or more developers as defined in claim 1, and
(ii) one or more couplers (A) and/or one or more couplers (B) and/or one or more couplers (C) as defined in claim 1, provided that it comprises at least one coupler (B) or (C).

21. A method of colouring hair comprising providing
(i) one or more developers as defined in claim 1, and
(ii) one or more couplers (A) and/or one or more couplers (B) and/or one or more couplers (C) as defined in claim 1, provided that it comprises at least one coupler (B) or (C), and
(iii) an oxidising agent
and applying (i), (ii) and (iii) to the hair to be coloured.

22. A method according to claim 21 in which the hair is substantially undamaged.

23. A method according to claim 21 in which the hair has previously been bleached, permed and/or previously coloured.

24. A method according to claim 21 in which part of the hair is substantially undamaged and part of the hair has previously been bleached, permed and/or previously coloured.

25. A hair colouring system which comprises
(i) one or more developers as defined in claim 1, and
(ii) one or more couplers (A) and/or one or more couplers (B) and/or one or more couplers (C) and optionally one or more compounds of the formulae XI to XIII herein, which comprises no other oxidative colouring agents and the system is capable of providing a wide spectrum of colour shades without the use of additional colouring agents.

26. A system according to claim 25 in which the couplers include only couplers (A) and/or (B) and/or (C).

27. A system according to claim 25 comprising couplers (A) and (B), optionally together with couplers of the formula 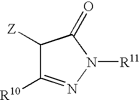
in which R¹⁰ and R¹¹ are selected from alkyl, cycloalkyl, alkenyl, cycloalkenyl, aryl, alkaryl, aralkyl, R′NHCOR, R′CONHR, SO₂R, SO₂NHR, R′SO₂R and R′SO₂NHR.

28. A kit, method or system according to any of claims 20 to 27 having any of the additional features set out in claims 2 to 19.

29. A composition, kit, method or system according to any preceding claim in which the developer (i) is selected from compounds which contain a single primary amine group and which react substantially only at the primary amine group under the conditions of hair colouring.

30. A hair colouring composition comprising N,N-dimethyl acetoacetamide and/or N,N-diethyl acetoacetamide.

31. A hair colouring composition comprising a compound of the formula 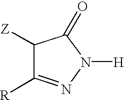
in which R is H, C₁₋₄ alkyl or phenyl.
